(19) United States
(12) Patent Application Publication
(10) Pub. No.: US 2019/0290330 A1
(43) Pub. Date: Sep. 26, 2019

(54) SURGICAL REPOSITIONING INSTRUMENT

(71) Applicants: Zbigniew Combrowski, Tuttlingen (DE); Alexander Henninger, Mühlheim (DE); Christel Paroth, Tuttlingen (DE)

(72) Inventors: Zbigniew Combrowski, Tuttlingen (DE); Alexander Henninger, Mühlheim (DE); Christel Paroth, Tuttlingen (DE)

(21) Appl. No.: 16/339,127

(22) PCT Filed: Oct. 4, 2017
(86) PCT No.: PCT/EP2017/075121

§ 371 Date: Apr. 3, 2019

(30) Foreign Application Priority Data
Oct. 4, 2016 (DE) 10 2016 118 718.6

(51) Int. Cl. A61B 17/70; A61F 2/46

(52) U.S. Cl. CPC A61B 17/708; A61B 2017/0256; A61F 2/4611

(57) ABSTRACT

In a surgical instrument including a reduction instrument (2) and an adapter (3) for a pedicle screw unit (4), the pedicle screw unit (4) including a pedicle screw (5), a tulip (6) and an insertion sleeve (7), the reduction instrument (2) is to have a first coupling point (8) and the adapter (3) is to have a second coupling point (9), it being possible for the first coupling point (8) and the second coupling point (9) to be connected to one another in a force-locking and/or form-fitting manner.

### TECHNICAL FIELD

The invention concerns a surgical instrument.

### STATE OF THE ART

Fixators such as pedicle screws, rods, cages (Cage is an intervertebral implant) are used to stabilize the spine.

Depending on the indication, open/minimally invasive surgery or in combination with a cage, for example, the pedicle screw on both sides of the implant must be removed after the implant has been inserted the spine, for example, first the cage, then other screws and the rods are inserted. However, both are also used as stand-alone solutions depending on the indication. In addition, various instruments such as retractors are used as spreading systems to create access for the insertion of a cage, compression and distraction instruments, straightening instruments (generic term: reduction devices), mostly via the pedicle screws, their tulips, insertion sleeves, extension shafts or extension instruments.

The vertebral bodies to be fixed can thus be distracted, compressed or straightened in order to achieve the optimum reduction of the vertebral bodies (this is referred to as the creation of sagittal balance).

For this purpose, very high forces must be generated and maintained with these instruments. This should be possible with the instrument(s) without loss of reduction, tension of the pedicle screw and rod constructs, for example.

Depending on the type of pedicle screw used, e.g. polyaxial, uniplanar or monoaxial, and depending on the spinal region (lumbar or thoracic), different rod shapes are also used, e.g. straight, lordotic or kyphotic. The screws must be repositioned parallel or at an angle.

For this purpose, a tension-free reduction and pre-fixing of the construct should be possible during reduction. Even during final fixation, the instrument(s) of the pedicle screws, for example, should allow sufficient axial freedom of movement so that the pedicle screws can be fixed as tension-free as possible with the rods. Tensions will otherwise lead to premature failure of the construct and a loss of reduction.

Depending on the indication and surgical technique, different screw shapes with different tulips, insertion sleeves, extension instruments and the corresponding reduction instrument sets are used by one manufacturer.

Retractor instruments for creating access for the insertion of a cage are also available in a wide variety of designs also. These often have the disadvantage that the retractor creates a tissue and muscle access for placing the cage and must be kept open during the operation. At the same time, a reduction instrument should be used to keep the disc space open and spread until the disc space for the cage has been removed and the cage inserted. Here the instruments often stand in the way of each other.

The disadvantage is that these retractor and reduction instruments consist of a variety of individual instruments and have a complex structure so that only experienced surgeons and surgical assistants can use these instruments without product specialists from the providers.

The U.S. Pat. Nos. 8,535,320, 8,906,034 and 8,100,828 show pedicle-based retractor and reduction instruments. The disadvantage of these is that they consist of a large number of individual parts for adjustment, so that all these systems do not have a bearing or pivot point in the tulip area in which the entire screw remains polyaxially movable and tensions occur on the pedicle screws and rods during reduction, pre-fixation and final fixation (depending on the indication, they are therefore mainly designed for polyaxial screws). Only the part of the screw that is screwed into the vertebra remains movable if it is a polyaxial screw. Depending on the indication, this is too little to avoid tension when using monoaxial or above all uniplanar screws, which is becoming more and more the trend with new surgical techniques in many indications such as spondeolisthesis, trauma, deformities and tumors. It is simpler, more stable and faster.

### SUMMARY OF THE INVENTION

The task of this invention is to overcome the disadvantages arising from the state of the art. In particular, a simple pedicle-based surgical device, consisting of a surgical instrument, i.e. the adapter for a pedicle screw unit, reduction instrument and retractor blade, is to be made available, which can be used openly and minimally invasively as a retractor and reduction device, and for cages and their access to screws and rod systems, for example, permits the most tension-free pre-fixing and/or final fixation possible without loss of reduction.

The characteristics disclosed herein lead to the solution of the task.

Advantageous designs are described.

The surgical instrument consists of a reduction instrument and an adapter for a pedicle screw unit. The pedicle screw unit usually consists of a pedicle screw, a tulip and an insertion sleeve.

The reduction instrument in this example is basically a spindle instrument that adjusts the adapter to another adapter. The spindle instrument is operated manually using a tool or a drive wheel.

The reduction instrument has a first coupling point and the adapter has a second coupling point, whereby the first coupling point and the second coupling point are connected to each other in a force-locking and/or form-fit manner are connected. The first coupling point of the reduction instrument has a first corrugation and the second coupling point of the adapter has a second corrugation. The first coupling point is cylindrical and the first corrugation is located on the outside of the coupling point. The second coupling point is sleeve-shaped and the second corrugation is arranged on an inner side of the second coupling point. An outer diameter of the first coupling point is smaller than an inner diameter of the second coupling point.

The adapter has at one end the second coupling point and at the other end a bearing shell. The bearing shell is preferably ring-shaped, whereby the ring shape can be completely closed or partially open. In this case, the bearing shell has a U-profile when viewed from above or below.

A connecting web extends between the second coupling point and the bearing shell. The connecting web is channel-shaped and has a bend of 70° to 100°, preferably 80° to 95° and even more preferably 90°.

The bearing shell forms an expansion towards the connecting web, which is suitable for polyaxially moving the tulip of the pedicle screw unit.

The bearing shell has two recesses arranged in a straight line at the other end of the connecting web. These recesses are shaped in such a way that they close off an unspecified oblong hole in the tulip towards the pedicle screw. Rods can be picked up here.

A slightly curved first rod is used kyphotically. A second slightly bent rod is used lordotically and a third unbent rod is used as required.

Furthermore, a retractor blade not shown in detail may be provided, which can be placed between the adapter and another adapter.

The surgical instrument mentioned in the invention can be used with any type of fixator, cage, pedicle screw, especially a bone screw or other implants or extracorporeal extensions of these implants.

The surgical instrument consists of the adapter for the pedicle screw unit, a retractor blade if necessary, and the reduction instrument. This should serve both as a retractor-spreading system for creating access and distraction of the intervertebral disc space, for inserting a cage, as well as a compression, distraction, straightening instrument for reduction, e.g. pedicle screws.

For this purpose, the surgical instrument should be able to generate and maintain very high forces. This should be possible with the surgical instrument without loss of reduction and avoiding unwanted tensions on the pedicle screw and rod construct, for example.

The reduction instrument allows the rod length to be determined using an integrated measuring scale before the rod is installed, the L+R spindle to provide an even traverse path and force transmission when the spindle is turned, and the reduction path to be checked during surgery with the surgical instrument or adapter. The thread is self-locking so that it holds the force and no loss of reduction can occur.

The surgical instrument should be as simple as possible.

The surgical instrument can be used for both open and minimally invasive surgery and allows simultaneous reduction on both sides of the spinal column, which makes parallel reduction possible in the case of a fracture, for example, and is also very important here.

With the surgical instrument, a reduction can be carried out parallel or at any angle over any number of vertebral segments.

The surgical instrument or the adapter of the surgical instrument is preferably provided at one end with the coupling point and at the other end with the ring-shaped bearing shell for e.g. a tulip or a pedicle screw. The ring-shaped bearing shell has two additional recesses arranged in a straight line. Depending on the application, these allow the user to place the respective rod in the desired position, but also in other places, e.g. along the pedicle screw. This influences the position of the bearing/pivot point for force application.

The closer the pivot point is to the respective rod, the easier the reduction is. For reduction, the screws must be able to slide easily with a minimum clearance to the rod, e.g. along the rod, i.e. axially, radially. The further away the pivot point is from the rod axis, the larger the lever arm, which leads to tilting and tension.

In between, the adapter or connecting bar is slightly shell-shaped and angled so that it does not obstruct the surgical field in conjunction with the reduction instrument. The bowl-shaped design is very wide open and allows a fixation during reduction that, for example, the pedicle screw can be aligned polyaxially outside the axis of the connecting bar.

The bearing shell is preferably closed in a ring shape and has an expansion on one inner side, which serves as bearing and pivot point. The pedicle screw can move freely in this polyaxial manner. A pedicle screw from the outside is preferred.

Depending on the type of pedicle screw used, e.g. polyaxial, uniplanar or monoaxial, and depending on the spinal area (lumbar or thoracic), different rod shapes are also used (e.g. straight, lordotic or kyphotic). The screws must therefore be repositioned parallel or at an angle.

For this purpose, it should be possible to re-construct, reposition or pre-fix the construct with as little tension as possible during reduction. Even during final fixation, the surgical instrument should allow the pedicle screws enough additional polyaxial freedom of movement so that the pedicle screws can be fixed as tension-free as possible with the rod(s). Otherwise tension will lead to premature failure of the construct as well as loss of reduction and associated revisions.

For this purpose, the ring-shaped bearing shell of the adapter of the surgical instrument is inserted preferably over the pedicle screws and up to the tulip rod area. The tulip is used to receive a rod that connects various pedicle screws intracorporeally and is fixed in the tulip with an ini.

It is also possible to take a picture of the extension of the tulip after a predetermined breaking point, i.e. the insertion sleeves, extension shafts or extension instruments.

Specially in minimally invasive applications, the surgical instrument or adapter can be inserted and placed through the same access of the pedicle screws. The screw access does not have to be extended for this purpose. Since the ring-shaped bearing shell is very thin-walled, there is no tension through tissue and muscles.

After the adapter has been inserted and placed, the reduction instrument is coupled only now. The advantage here is the fine toothing or ribbing at both coupling points and the corresponding play of both parts. This avoids tensions.

It is also conceivable to have a rigid connection between the two instead of the coupling points, but this would make it more difficult to insert and place the instrument with the disadvantages mentioned above.

The coupling point in the adapter is shown as a cylindrical bore with a fine toothing or fine ribbing and the coupling point of the reduction instrument as a mandrel with a fine toothing or ribbing, which interact with each other during play.

However, the coupling point can also, for example, have either a face serration or corrugation, it can be conical or shaped as a ball head with a type of serration which can transmit large forces.

A simple plug-in connection is also possible, which can only be fixed in any position with a screw, for example.

The retractor blade should preferably be manufactured as a simple sheet metal part and can preferably be adapted based on pedicle screws.

At one end the retractor blade has a handle and at the other end a holder, with which it can be fixed in the pedicle screw and an angled tongue, which serves to keep away muscle mass. Preferably only one retractor blade is used, but there can also be two. This is not described in detail. A mirrored version would be used for this purpose is not described in more detail. A mirrored variant would be attached to the other pedicle screw to form the access channel between the pedicle screws. It is conceivable that the two retractor blades can then be adjusted in a fan shape and are each provided with a catch, which allows better fixation.

It is advantageous to use two retractor blades as a retractor spreading system to create access and distraction, the intervertebral disc space, up to the insertion of a cage. After the surgical instruments or adapters have been placed on the pedicle screws and coupled to the reduction instrument, the two retractor blades are fixed in the pedicle screws. In this way, the adapters are additionally secured against removal so that they cannot slip during reduction and there would be no loss of reduction.

The retractor blade can have any shape, such as a tube almost round, cylindrical or conical and does not have to be closed (e.g. half open like a channel) and is shown in the present version as a retractor blade. The pedicle screws have certain axialities. This allows the retractor blade to be placed in the desired position.

Preferably, the holder is cylindrical like a rod and shaped in the diameter of the rod. In the version shown, it is shown as a stable sheet metal, whereby the surfaces to which it is fixed are preferably rounded. Cylindrical like a bar and shaped in the diameter of the bar. The fixing is preferably done with the Ini. However, a fixation in the surgical instrument is also conceivable. A stop or a coupling to the surgical instrument for the retractor is also possible.

The surgical instrument can be used in open and MIS surgery and preferably consists of several parts, in order to be as tension-free as possible and preferably via an equal access of the pedicle screws can be inserted tissue-sparingly, so that the pedicle screw remains polyaxially movable during reduction even under very high forces. This allows tension-free pre-fixing and final fixation without loss of reduction of the construct.

The adapter is available in different lengths, preferred lengths are 50 to 200 mm and even more preferred lengths are 60 to 130 mm.

The second coupling point of the adapter has a groove which interacts with a notch at the coupling point of the reduction instrument in such a way that the two coupling points can only be detached from each other by selective pulling. This prevents the instrument from falling out unintentionally.

As mentioned above, the coupling point of the adapter is preferably located on the inside and the coupling point of the reduction instrument on the outside. This can also be the other way round.

The corrugations and the serrations can vary in fineness and distance and are usually arranged in a 360° circle.

### FIGURE DESCRIPTION

Further advantages, features and details of the invention result from the following description of preferred execution examples as well as from the drawings; these show in:

FIG. 1 shows a perspective view of a surgical instrument according to the invention;

FIG. 2 a front view of the surgical instrument according to FIG. 1;

FIG. 3 a side view of the surgical instrument according to FIG. 1;

FIG. 4 a perspective view of an adapter for use in the surgical instrument shown in FIG. 1;

FIG. 5 a side view of the adapter according to FIG. 4;

FIG. 6 a rear view of the adapter according to FIG. 4;

FIG. 7 another perspective view of the adapter according to FIG. 4.

### DETAILED DESCRIPTION

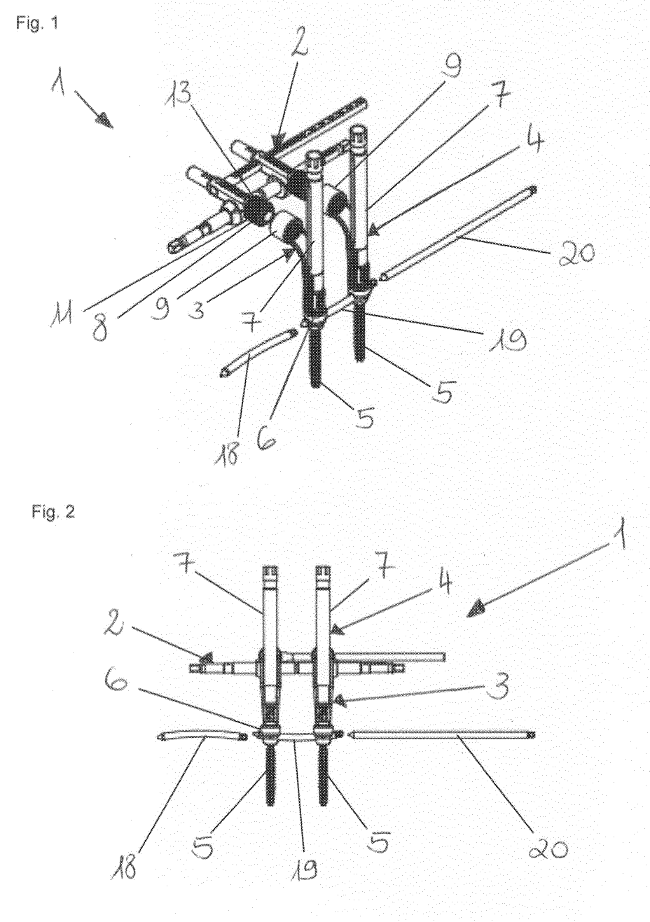

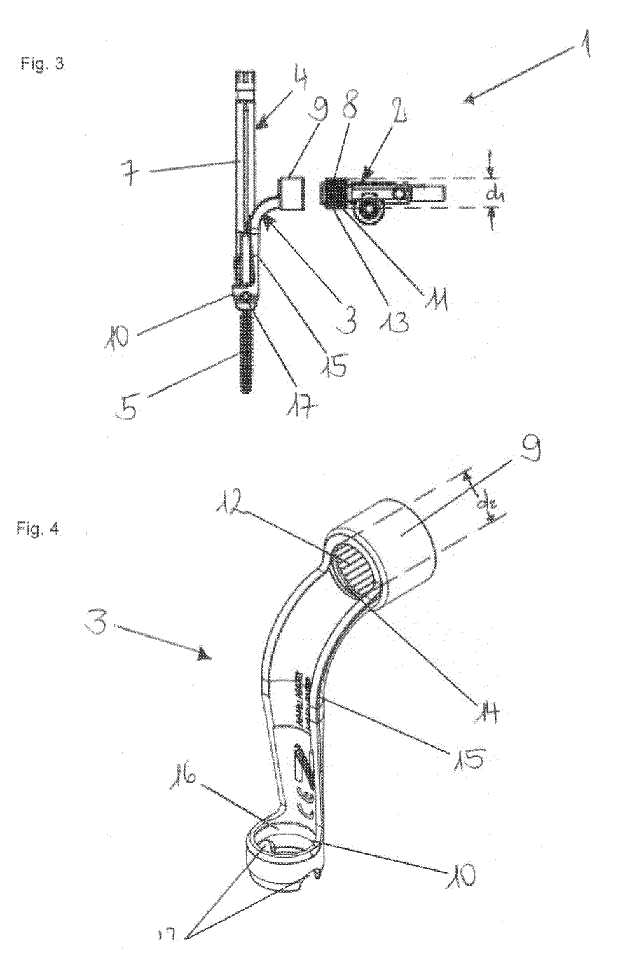

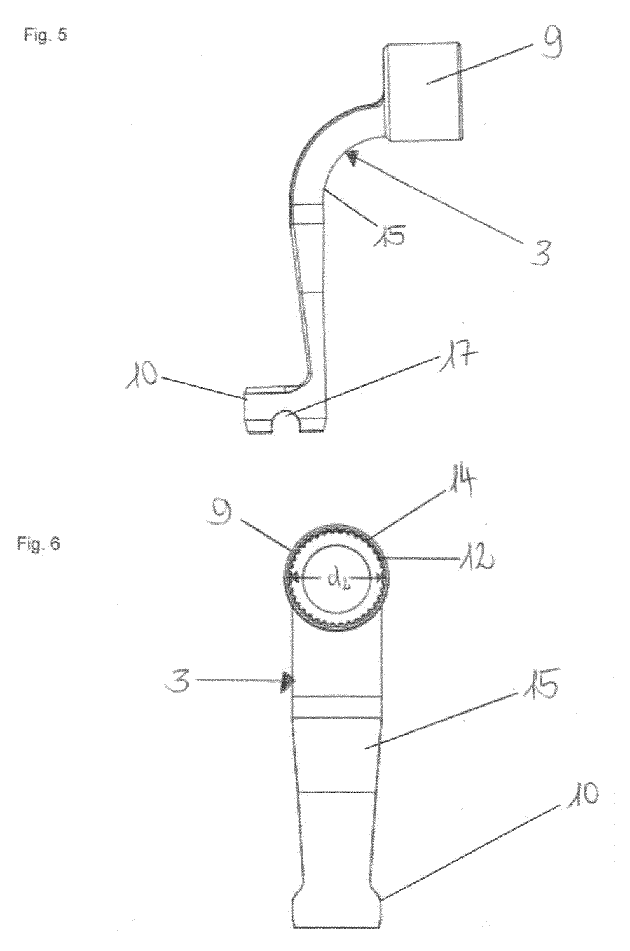

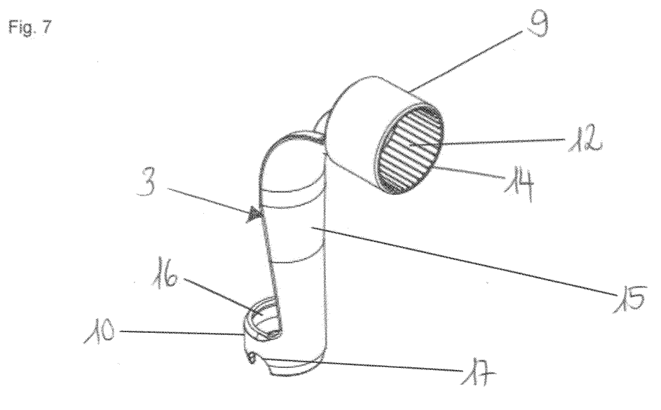

FIG. 1 shows a perspective view of a surgical instrument 1. The surgical instrument 1 consists of a reduction instrument 2 and an adapter 3 for a pedicle screw unit 4. The pedicle screw unit 4 comprises a pedicle screw 5, a tulip 6 and an insertion sleeve 7.

The reduction instrument 2 in this design example is basically a spindle instrument that connects adapter 3 to another adapter adjusts or configures. The spindle instrument is operated manually via a tool or a drive wheel. The reduction instrument 2 has a first coupling point 8 and the adapter 3 has a second coupling point 9, whereby the first coupling point 8 and the second coupling point 9 are connected to each other in a force-locking and/or form-fit manner.

The adapter 3 has at one end the second coupling point 9 and at the other end a bearing shell 10. The bearing shell 10 is preferably ring-shaped, whereby the ring shape can be completely closed or partially open. In this case, the bearing shell 10 can have more or less a U-profile when viewed from above or below.

The first coupling point 8 of the reduction instrument 2 has a first corrugation 11 and the second coupling point 9 of the adapter 3 has a second corrugation 12.

The first coupling point 8 is cylindrical and the first corrugation 11 is arranged on an outside 13 of the coupling point 8.

The second coupling point 9 is sleeve-shaped and the second corrugation 12 is arranged on an inside 14 of the second coupling point 9.

An outer diameter d1 of the first coupling point 8 is smaller than an inner diameter d2 of the second coupling point 9.

A connecting web 15 extends between the second coupling point 9 and the bearing shell 10. The connecting web 15 is channel-shaped and has a bend of 70° to 100°, preferably 80° to 95° and even more preferably 90°.

The bearing shell 10 forms a widening 16 towards the connecting web 15, which is suitable to accommodate the tulip 6 of the pedicle screw unit 4 in a polyaxially movable manner.

The bearing shell 10 has two recesses 17 arranged in a straight line at the other end of the connecting web 15. These recesses 17 are shaped in such a way that they close off an oblong hole of the tulip 6, which is not shown in detail, towards the pedicle screw 5.

A slightly curved first rod 18 is used kyphotically. A second slightly bent rod 19 is used lordotically and a third unbent rod 20 is used as required.

Furthermore, a retractor blade not shown in detail can be provided, which can be placed between adapter 3 and another adapter.

### REFERENCE CHARACTER LIST

1. surgical instrument
2. Reduction instrument
3. adapters
4. Pedicle screw unit
5. Pedicle screw
6. Tulip
7. Insertion sleeve
8. Coupling point
9. Coupling point
10. Bearing shell
11. Ribbing
12. Ribbing
13. Outside
14. Inside
15. Connecting bridge
16. Expansion
17. Recess
18. Rod
19. Rod
20. Rod
d1—Outer diameter
d2—Inner diameter

1. Surgical instrument consisting of a reduction instrument (2) and an adapter (3) for a pedicle screw unit (4), wherein the pedicle screw unit (4) comprises a pedicle screw (5), a tulip (6) and an insertion sleeve (7),
wherein the reduction instrument (2) has a first coupling point (8) and the adapter (3) has a second coupling point (9), the first coupling point (8) and the second coupling point (9) being connectable to one another in a force-locking and/or form-fitting manner.

2. Surgical instrument according to claim 1, wherein the adapter (3) has at one end the second coupling point (9) and at the other end comprises a bearing shell (10).

3. Surgical instrument according to claim 1, wherein the first coupling point (8) has a first corrugation (11) and the second coupling point (9) has a second corrugation (12).

4. Surgical instrument according to claim 3, wherein the first coupling point (8) is cylindrically shaped and the first corrugation (11) is arranged on an outside (13) of the coupling point (8).

5. Surgical instrument according to claim 3, wherein the second coupling point (9) is sleeve-shaped and the second corrugation (12) is arranged on an inside (14) of the second coupling point (9).

6. Surgical instrument according to claim 4, wherein an outer diameter (d1) of the first coupling point (8) is smaller than an inner diameter (d2) of the second coupling point (9).

7. Surgical instrument according to claim 2, wherein a connecting web (15) extends between the second coupling point (9) and the bearing shell (10).

8. Surgical instrument according to claim 7, wherein the connecting web (15) is channel-shaped.

9. Surgical instrument according to claim 7, wherein the connecting web (15) has a bend of 70° to 100°.

10. Surgical instrument according to claim 2, wherein the bearing shell (10) forms an expansion (16) towards the connecting web (15), suitable for receiving the tulip (6) of the pedicle screw unit (4) in a polyaxially movable manner.

11. Surgical instrument according to claim 2, wherein the bearing shell (10) has two recesses (17) arranged in a straight line at the other end of the connecting web (15).

12. Surgical instrument according to claim 2, wherein the bearing shell (10) is annular in shape, the annular shape being able to be completely closed or partially open, whereby the bearing shell (10) forms a U-profile.

13. Surgical instrument according to claim 1, wherein a pedicle screw (5) based retractor blade is provided, which can be arranged between the adapter (3) and a further adapter.

14. Surgical instrument according to claim 9, wherein the bend is 80° to 95°.

15. Surgical instrument according to claim 9, wherein the bend is 90°.
